(19) United States
(12) Patent Application Publication
(10) Pub. No.: US 2023/0238123 A1
(43) Pub. Date: Jul. 27, 2023

(54) AI-BASED APPLICATION TO PREDICT APPOINTMENT ADHERENCE IN PEDIATRIC SETTINGS

(71) Applicant: West Virginia University Board of Governors on Behalf of West Virginia University, Morgantown, WV (US)

(72) Inventors: Janani Narumanchi, Morgantown, WV (US); Naveena Yanamala, Morgantown, WV (US); Tarachandra Narumanchi, Morgantown, WV (US)

(21) Appl. No.: 18/157,289

(22) Filed: Jan. 20, 2023

Related U.S. Application Data
(60) Provisional application No. 63/301,547, filed on Jan. 21, 2022.

(51) Int. Cl. G16H 40/20; G16H 50/20; G06Q 10/1093

(52) U.S. Cl. CPC G16H 40/20; G16H 50/20; G06Q 10/1095

(57) ABSTRACT

An appointment scheduling system configured to predict the likelihood of a missed appointment during the scheduling process, thereby minimizing missed appointments and improving access to care is discussed. In particular, artificial intelligence/machine-learning techniques are used to more efficiently utilize a provider's time and a patient's needs and to help optimize scheduling at healthcare facilities. A predictive model can aid in prioritizing the design and implementation of interventions that may improve efficiency towards more timely access to care at healthcare facilities. In particular, patients can be provided with an earliest appointment, closer to his or her home. In addition, patient and provider satisfaction with scheduling services can be improved.

### CROSS-REFERENCE TO RELATED APPLICATIONS

This application claims the benefit of and priority to U.S. Provisional Patent Application No. 63/301,547 filed on Jan. 21, 2022, entitled “AI-BASED APPLICATION TO PREDICT APPOINTMENT ADHERENCE IN PEDIATRIC SETTINGS,” the contents of which are incorporated by reference in their entirety herein.

### BACKGROUND

There is increasing frustration amongst both patients and providers regarding the scheduling and wait times of patients to be seen by clinicians. Administration demands that patients will be seen as soon as possible after a referral is made. Even when the provider's schedules are filled appropriately, this demand is difficult to meet for multiple reasons. In some cases, patients fail to show-up for their appointments. This causes provider frustration as well delaying care for patients who are still waiting for an appointment.

### BRIEF DESCRIPTION OF THE DRAWINGS

Many aspects of the present disclosure can be better understood by referring to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a drawing of an example schema of the appointment scheduling system according to various embodiments of the present disclosure.

FIG. 2 is a schematic block diagram of a networked environment according to various embodiments of the present disclosure.

FIG. 3 is a flowchart illustrating one example of functionality implemented as portions of the appointment scheduling system executed in a computing environment in the networked environment of FIG. 2 according to various embodiments of the present disclosure.

FIG. 4 is a flowchart illustrating one example of functionality implemented as portions of the appointment scheduling system executed in a computing environment in the networked environment of FIG. 2 according to various embodiments of the present disclosure.

FIG. 5 is a schematic block diagram that provides one example illustration of a computing environment employed in the networked environment of FIG. 2 according to various embodiments of the present disclosure.

### DETAILED DESCRIPTION

The present disclosure relates to an appointment scheduling system configured to predict the likelihood of a missed appointment during the scheduling process, thereby minimizing missed appointments and improving access to care in accordance to various embodiments of the present disclosure. In particular, artificial intelligence/machine-learning techniques are used to more efficiently utilize a provider's time and a patient's needs and to help optimize scheduling at healthcare facilities. According to various embodiments, predictive modeling techniques aid in prioritizing the design and implementation of interventions that may improve efficiency towards more timely access to care at healthcare facilities. In particular, patients can be provided with an earliest appointment, closer to his or her home. In addition, patient and provider satisfaction with scheduling services can be improved.

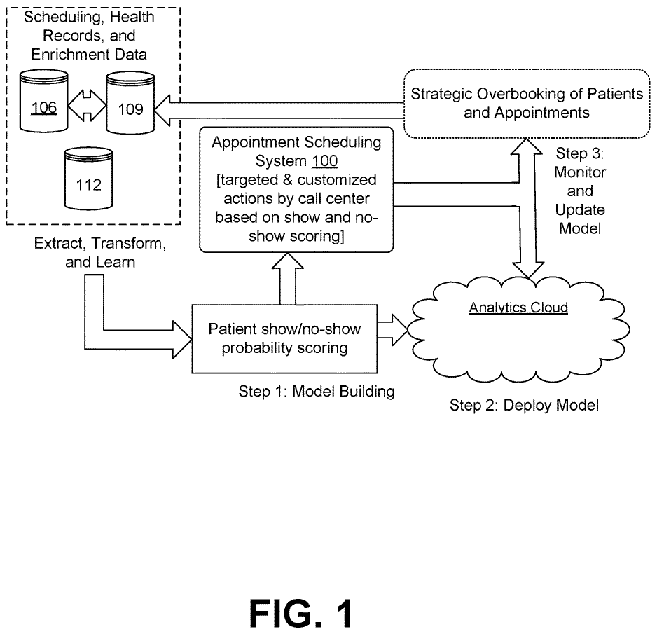

FIG. 1 illustrates an example overview of the appointment scheduling system 100 in accordance of various embodiments. In some examples, the appointment scheduling system 100 can be used to help with the live scheduling via a phone call, a chat interface, and/or other type of interface that facilitates live scheduling. In some examples, the appointment scheduling system 100 can be a standalone system and/or can be implemented in applications, such as, for example, “MyChart” scheduling as well as “Open scheduling” and “Direct scheduling” features that allow patients to schedule their own appointments.

As shown in FIG. 1, a no-show/cancellation predictor model can be created and updated using data associated with past appointments. The no-show/cancellation predictor model can be trained to output a result denoting a “show” or a “no-show” along with a probability score based on a variety of factors, included, for example, demographics, a time between receiving a referral and a date of appointment, weather, lifestyle, method of transportation, drive time and traffic, custom events/holidays, clinic specialties, insurance type, patient family specific variables, and/or other factors.

AI-enabled technologies and solutions have been shown decreased wait time in clinic appointments solutions. For example, AI-enabled technologies are known to be used in the analysis of electronic health records (EHR) 106 and scheduling data 109 to predict missed appointments in community health centers, appointment non-adherence, to decide patient scheduling as well as their treatment options in cancer centers, identifying patients who are likely to skip MRI scans and/or procedures, and to predict hospital re-admissions. These known techniques suggest improved patient/provider satisfaction with options tailored specifically for each facility, based on machine learning analysis of patient preferences. For example, different predictive modeling methodologies, namely logistic regression, naïve Bayes classifier, and artificial neural networks have been previously used to develop models that informed actionable measures from scheduling practices to reminder systems, redesign operations and policies, and to predicting risk-adjusted panel sizes for physicians, which all resulted in boosting patient/provider satisfaction.

According to various embodiments, a data analytic software (e.g., Tableau®) can be used to gather data on clinic performances, including slot utilization, wait time and no-show rates. All this data provides an opportunity for making improvements.

Call centers can be used to schedule appoints for patients seen in both primary care and specialty clinics. For examples, the pediatric pod of the call center for West Virginia University (WVU) Medicine has about nine people who handle, on average, 554 calls per day and were estimated to answer over a one million calls by end of 2019. They spend an average of five minutes (sometimes longer for new patients) on the phone to offer the scheduling party appointment choices for a given appointment. The goal of the call center representative is to provide the earliest available appointment (at the time of the call).

The call center for WVU Medicine directly schedules patients using a scheduling system such as, for example, EPIC®. There are scheduled quarterly updates of Epic® to help optimize and streamline some aspects of scheduling. However, there are still challenges faced with scheduling including: difficulty making appointments within days after the referral, a decreased show rate, and not being able to accommodate the patients in a clinic close to their residence. Current data doesn't give insight as to why this is happening and how the whole process can be improved.

According to various embodiments, the appointment scheduling system 100 of the present disclosure uses a variety of factors that can be derived from scheduling data 109, health records data 106, and enrichment data 112. These factors can include demographic information, patient family specific variables (e.g., parent/guardian education, parent/guardian occupation, etc.), demographics, duration between date of referral and date of anticipated appointment, weather, lifestyle, method of transportation, drive time and traffic, custom events/holidays, clinic specialties, insurance type and/or other factors to provide insight to patterns of behavior with regard to appointment attendance. According to various embodiments, predictive analytics is used to develop algorithm(s) that will aid in informing schedulers on the optimal location for the patient to schedule an appointment for referral, along with recommendations on how to schedule patients with high no-show rates (e.g., strategically double book the slot or give only half the usual time for appointment) while they are still on the phone with the family. This should improve the ability to give the earliest appointment (e.g., within days) after receiving a referral, thereby decreasing no show rate, and accommodating the patients in a clinic close to their residence. According to various embodiments, patient and provider satisfaction are improved with scheduling services in clinic.

According to various embodiments, the no-show predictor model of appointment scheduling system 100 of the present disclosure can work in conjunction with the various appointment reminder systems. In various embodiments, the number of calls, emails, and texts that were sent to the patients, as well as using the responses to those actions, can be used as additional inputs into the models for better accuracy. AI can provide automated decision-making system giving personalized probabilities per patient and incorporate the P4 medicine (e.g., predictive, preventive, personalized and participatory) into practice.

Cancelled appointments, or “no-shows,” are unfortunately common. Missed appointments can have a huge impact on our healthcare practice. Not able to provide the treatment the patient needs, wasted provider and staff prep time, lost revenue, a hit on the quality of care for other patients and not to mention patient/provider satisfaction—the holy grail of any healthcare practice. No matter how dedicated and organized a clinic may be, it's impossible to guarantee that every patient who makes an appointment will keep it. There could be multiple reasons and human factors that could be influencing no-shows at healthcare clinics. Previous research indicates patients who miss appointments tend to be of lower socio-economic status, often have a history of failed/no-show appointments, government-provided health benefits, and psychosocial problems. They are also less likely to understand the purpose of the appointment.

No-show and cancellation rates also increase with increasing time between scheduling and the actual appointment. In addition to forgetting appointments, issues such as trouble getting off work, finding childcare, transportation, and cost also factored into patients that failed to show for an appointment. The reasons for no-show or otherwise cancelled appointments may include, for example, long wait times between a referral and a scheduled appointment and accessibility of clinic locations in relation to patient's residence. It is also important to note that patients who fail to show up to their appointments interrupt efforts to provide continuity of care. Therefore, predicting and preventing missed appointments can potentially improve access to care as well as increased patient/provider satisfaction. The ultimate goal of the present disclosure is to improve the scheduling of clinic appointments for all families who seek care in any healthcare facility.

The AI-based solutions of the appointment scheduling system 100, such as machine-learning based modeling approaches, are configured to predict the likelihood a patient will adhere or show up for an appointment. The appointment scheduling system 100 can be used to further aid in automatically scheduling new appointments instantly and effectively for patients in need of care. In addition, the wait time in scheduling an appointment from the time of the referral can be decreased, while keeping in mind family preference and needs. Further, the embodiments of the present disclosure can maximize the efficiency of call center staff.

Data used to train the predictive models of the appointment scheduling system may include census demographics, weather, lifestyle, method of transportation, drive time and traffic, custom events/holidays, clinic specialties, insurance type and/or other factors. This data can be gathered and analyzed to improve understanding of the complexity of appointment adherence of patients. According to various embodiments, a predictive modeling algorithm is developed using the obtained data to calculate a no-show score for each patient. Schedulers and other healthcare providers can then use these scores to look at their weekly schedule and find which patients are at high risk for not showing up for their appointments and identify best ways to use appointment slots.

Clinics have different population characteristics, specialties, and patient demographics; thus, it is important to note that appointment adherence varies across geographic sites. For example, specialty clinics might have higher missed appointment rates than the ones providing acute or general primary care. Thus, it becomes important for us to perform an exploratory analysis to identify some of these factors specific to healthcare clinics.

To gain further insights into factors/reasons and understand what could be influencing no-show rates and other patient related patterns the present disclosure relies on a variety factors including, but not limited to date/time of the clinic visit, local weather conditions around their visits, patient's zip code, travel distance to/from the clinic, education/literacy level, parental education/literacy for pediatric patients, age of parent/caregiver, insurance/employment status to extrapolate economic status that might impact their transportation needs, past history of (no-)show rates for their appointments.

In various embodiments, (un-)supervised learning approaches are used to identify variables or factors that are significantly associated with appointment adherence. The dataset can be split into training and test sets. Logistic regression or other supervised learning approaches including, traditional, ensemble- and deep learning-based techniques, to develop a predictive model for the binary outcome (e.g., show or a no-show) of whether each patient arrived or failed to show for their appointment.

According to various embodiments, the generated predictive model of the appointment scheduling system 100 is used to estimate a no-show probability score for a visit based on two outcomes, either show or no-show, excluding other modifiers such as late shows. In order to translate the probability to a predicted show or no-show, a threshold will be determined to clearly separate the two classes.

According to various embodiments, an overbooking model can be implemented into the scheduling system 100 to consider individual no-show prediction rates. In some embodiments, the appointment scheduling system 100 provides recommendations to schedulers including recommendations for an optimal location for the patient to be scheduled and probability of “no-shows” to their upcoming appointment. In addition, the appointment scheduling system 100 can generate and provide recommendations that recommend how to schedule such a patient (e.g., strategically double book the slot or give only half the usual time for appointment or offer a virtual visit via MyChart video or telephone visit or consider home health visits if connectivity for virtual visits is also a limiting factor for appointment attendance) while they are on the phone with the family.

The present disclosure can improve existing technologies by redesigning and/or optimizing current scheduling processes to reduce the number of no-show appointments. In addition, the no-show prediction models of the present disclosure can be implemented into scheduling systems (e.g., phone calls, “My chart” scheduling, “Open scheduling” and “Direct scheduling” features that allow patients to schedule their own appointments, etc.) as a decision support systems that would identify patients with a high risk of appointment no-show so that appointments with high risk of no-show may be strategically double booked, or patients with high risk of no-show may be reminded more rigorously.

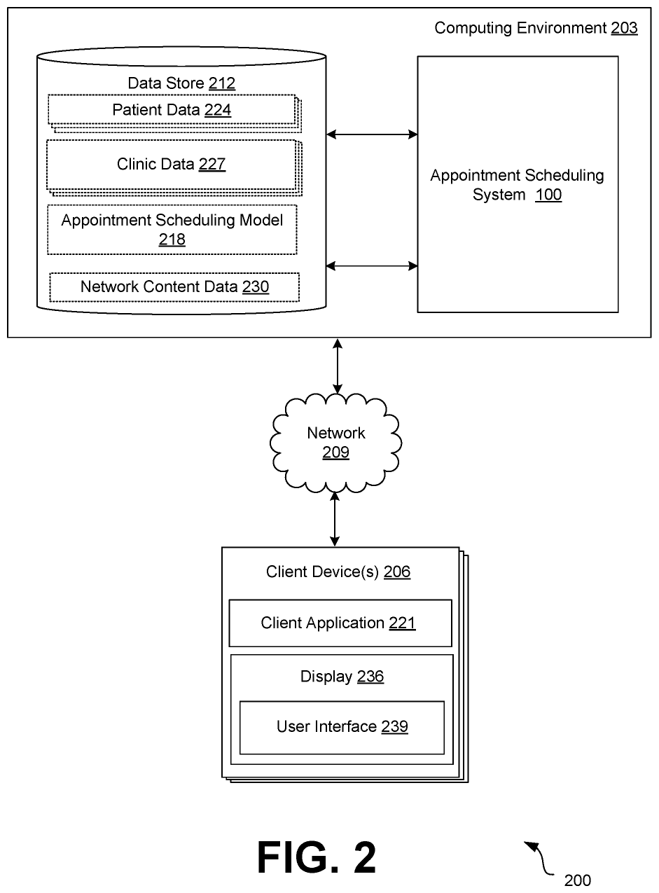

In the following discussion, a general description of the system and its components is provided, followed by a discussion of the operation of the same. With reference to FIG. 2, shown is a networked environment 200 according to various embodiments. The networked environment 200 includes a computing environment 203 and a client 206, which are in data communication with each other via a network 209. The network 209 can include wide area networks (WANs), local area networks (LANs), personal area networks (PANs), or a combination thereof. These networks can include wired or wireless components or a combination thereof. Wired networks can include Ethernet networks, cable networks, fiber optic networks, and telephone networks such as dial-up, digital subscriber line (DSL), and integrated services digital network (ISDN) networks. Wireless networks can include cellular networks, satellite networks, Institute of Electrical and Electronic Engineers (IEEE) 802.11 wireless networks (i.e., WI-FI®), BLUETOOTH® networks, microwave transmission networks, as well as other networks relying on radio broadcasts. The network 209 can also include a combination of two or more networks 209. Examples of networks 209 can include the Internet, intranets, extranets, virtual private networks (VPNs), and similar networks.

The computing environment 203 may comprise, for example, a server computer or any other system providing computing capability. Alternatively, the computing environment 203 may employ a plurality of computing devices that may be arranged, for example, in one or more server banks or computer banks or other arrangements. Such computing devices may be located in a single installation or may be distributed among many different geographical locations. For example, the computing environment 203 may include a plurality of computing devices that together may comprise a hosted computing resource, a grid computing resource, and/or any other distributed computing arrangement. In some cases, the computing environment 203 may correspond to an elastic computing resource where the allotted capacity of processing, network, storage, or other computing-related resources may vary over time.

Various applications and/or other functionality may be executed in the computing environment 203 according to various embodiments. Also, various data is stored in a data store 212 that is accessible to the computing environment 203. The data store 212 may be representative of a plurality of data stores 212 as can be appreciated. The data stored in the data store 212, for example, is associated with the operation of the various applications and/or functional entities described below.

The components executed on the computing environment 203, for example, include an appointment scheduling system 100, and other applications, services, processes, systems, engines, or functionality not discussed in detail herein. The appointment scheduling system 100 is executed to train and update an appointment prediction model 218 to predict a likelihood of whether a particular patient will show for a given appointment. The appointment scheduling system 100 can further interact with the client application 221 on the client device and/or other devices to obtain scheduling data 109 and patient data 224 that can be used as inputs that can be used as inputs that are applied to a trained appointment prediction model 218. For example, a client application 221 executed on the client device 206 may send an appointment prediction request to the appointment scheduling system 100. The request may include patient data, appointment data, and/or other information that can be applied to the trained appointment prediction model 218. The output of the appointment prediction model 218 can comprise a binary score that indicates whether a no-show or a “show” is predicted for the given patient/appointment. The appointment scheduling system 100 can send a result back to the client application 221 to allow the scheduler the opportunity to schedule an appointment based on the result. In some examples, the appointment scheduling system 100 can generate and provide a recommendation for the scheduling providing a recommendation of how to schedule the appointment for the particular patient based on the result of the appointment prediction model 218.

The data stored in the data store 212 includes, for example, patient data 224, clinic data 227, an appointment prediction model 218, network content data 230, and potentially other data. The patient data 224 includes known data about the particular patient, including, for example, demographics, lifestyle, method of transportation, address, insurance type, specific variables related to the appointment including appointment type and prior appointment data statistics, patient family specific variables (e.g., parent/guardian ages, parent/guardian education, parent/guardian education, who normally brings patient to appointments, etc.), and/or other data. The clinic data 227 can include for example clinic specialties, appointment types, average appointment lengths for appointment types, and/or other factors. In various examples, the patient data 224 and the clinic data 227 can further include information obtained from the scheduling data 109, health records data 106, enrichment data 112, and/or other data.

The appointment prediction model 218 includes, for example, a logistic regression classifier, a random forest classifier, a decision tree classifier, a XGBoost classifier, a multi-layer perceptron classifier, a recurrent neural network, a feed-forward neural network, a label-specific attention network, and/or any other type of trained model as can be appreciated.

The network content data 230 includes various data employed by the appointment prediction system 100 in generating user interfaces 239, and/or other network pages. In some embodiments, the network content data 233 includes hypertext markup language (HTML), extensible markup language (XML), cascading style sheets (CSS), images, text, audio, video, templates, and/or other data.

The client 206 is representative of a plurality of client devices that may be coupled to the network 209. The client 206 may comprise, for example, a processor-based system such as a computer system. Such a computer system may be embodied in the form of a desktop computer, a laptop computer, personal digital assistants, cellular telephones, smartphones, set-top boxes, music players, web pads, tablet computer systems, game consoles, electronic book readers, smartwatches, head mounted displays, voice interface devices, or other devices. The client 206 may include a display 236. The display 236 may comprise, for example, one or more devices such as liquid crystal display (LCD) displays, gas plasma-based flat panel displays, organic light emitting diode (OLED) displays, electrophoretic ink (E ink) displays, LCD projectors, or other types of display devices, etc.

The client 206 may be configured to execute various applications such as a client application 221 and/or other applications. The client application 221 may be executed in a client 206, for example, to access network content served up by the computing environment 203 and/or other servers, thereby rendering a user interface 239 on the display 236. To this end, the client application 221 may comprise, for example, a browser, a dedicated application, etc., and the user interface 239 may comprise a network page, an application screen, etc. The client 206 may be configured to execute applications beyond the client application 221 such as, for example, email applications, social networking applications, word processors, spreadsheets, and/or other applications.

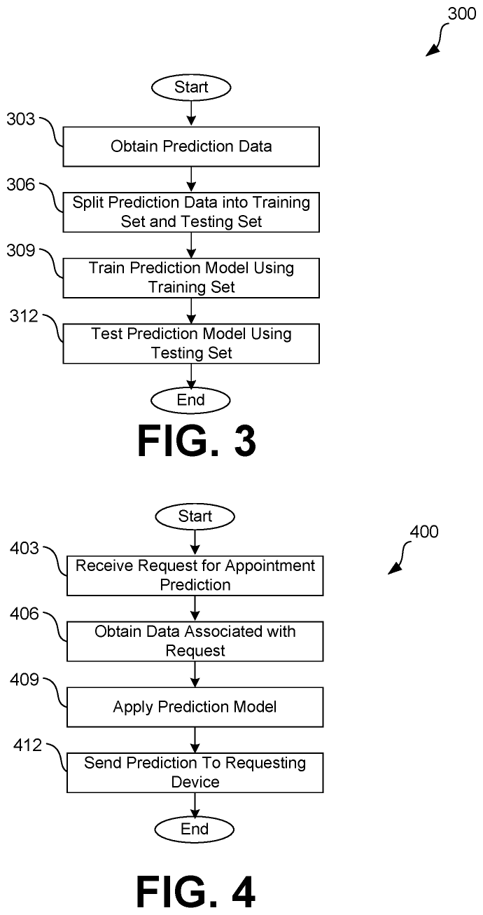

Referring next to FIG. 3, shown is a flowchart 300 that provides one example of the operation of a portion of the appointment scheduling system 100 according to various embodiments. It is understood that the flowchart of FIG. 3 provides merely an example of the many different types of functional arrangements that may be employed to implement the operation of the portion of the appointment scheduling system 100 as described herein. As an alternative, the flowchart 300 of FIG. 3 may be viewed as depicting an example of elements of a method implemented in the computing environment 203 (FIG. 2) according to one or more embodiments.

Beginning with box 303, the appointment scheduling system 100 obtains prediction data. The prediction data can include historical appointment data for past patient appointments. The historical appointment data can comprise patient data 224, clinic data 227, weather, traffic, travel time, and/or other data as can be appreciated. In experiments, the appointment scheduling system 100 used historical appointment data covering multiple years. In various examples, the predication data can include information obtained from the scheduling data 109, health records data 106, enrichment data 112, and/or other data.

At box 306, the appointment scheduling system 100 splits the obtained prediction data into a training set of data and a testing set of data. For example, the training set of data can comprise the data that is used to train the prediction model 218 and the testing set of data is the data used to test the accuracy of the predication model 218.

At box 309, the appointment scheduling system 100 trains the appointment prediction model 218. As discussed, the appointment prediction model 218 corresponds to a supervised learning approach to predicting a likelihood of whether a patient will show or fail to show for a given appointment. In various examples, the appointment prediction model 218 comprises a supervised machine learning models such as, for example, a logistic regression classifier, a random forest classifier, a decision tree classifier, a XGBoost classifier, a multi-layer perceptron classifier, a recurrent neural network, and/or any other type of trained model as can be appreciated.

At box 312, the appointment scheduling system 100 tests the accuracy of the appointment prediction model 218 using the testing set of data. For example, the corresponding inputs for the appointment prediction model 218 can be applied to the trained appointment prediction model 218 and the predict results can be compared with the actual results to determine accuracy of the model. Thereafter, this portion of the process proceeds to completion.

Referring next to FIG. 4, shown is a flowchart 400 that provides one example of the operation of a portion of the appointment scheduling system 100 according to various embodiments. It is understood that the flowchart 400 of FIG. 4 provides merely an example of the many different types of functional arrangements that may be employed to implement the operation of the portion of the appointment scheduling system 100 as described herein. As an alternative, the flowchart 400 of FIG. 4 may be viewed as depicting an example of elements of a method implemented in the computing environment 203 (FIG. 2) according to one or more embodiments.

At box 403, the appointment scheduling system 100 can receive a request from a client device 206 associated with a scheduler or other entity. The request can be a request to determine a likelihood of whether a patient will show or fail to show for a particular appointment at a given healthcare clinic. The request can include patient data 224, clinic data 227, appointment data (e.g., proposed day/time, appointment type, etc.), and/or other data associated with a given appointment. In some examples, the request includes an identification of the type of healthcare clinic associated with the request.

At box 406, the appointment scheduling system 100 can obtain data associated with the request. In some examples, obtaining the data includes extracting the required input data from the patient data 224, clinic data 227, appointment data, and/or other data. In some examples, external data, such as, weather, traffic, and/or other data can be obtained from various external sources. For example, the appointment scheduling system 100 may contact an external weather data source to obtain a prediction of the weather for a given day/time associated with the appointment. Similarly, the appointment scheduling system 100 can contact an external traffic data source to obtain a predication of traffic for a given day/time associated with the appointment based at least in part on the patient address and the clinic address. In various examples, the appointment scheduling system 100 can interface with other entities to obtain the required information through the use of application programming interface (API) calls.

At box 409, the appointment scheduling system 100 obtains inputs associated with the appointment request and applies the inputs to the trained appointment prediction model 218. The output of appointment prediction model 218 includes a score that indicates a show or no-show prediction associated with the inputted variables. In some examples, a plurality of different appointment prediction models are trained for different types of healthcare clinic such that a first appointment prediction model is trained for a first type of healthcare clinic and a second appointment prediction model is trained for a second type of healthcare clinic. As such, in some examples, the appointment scheduling system 100 selects a particular appointment prediction model 218 from the plurality of appointment prediction models based at least in part on the type of healthcare clinic associated with the appointment request.

At box 412, the appointment scheduling system 100 generates a prediction notification based at least in part on the score from the appointment prediction model 218 and sends the prediction notification to the requesting device 206. In some examples, the prediction notification may include a recommendation on how to schedule a given appointment for the patient based at least in part on the type of healthcare clinic, type of appointment; patient information, and/or other factors. In various examples, the prediction notification can comprise a user interface 239 and/or a user interface element that is rendered on a user interface 239 that is rendered on the client device 206. In various examples, the appointment scheduling system 100 generates user interface code corresponding to the user interface 239 and/or user interface element and transmits the user interface code to the client device 206. In this situation, the client application of the client device 206 generates and causes the user interface 239 and/or user interface element to be rendered on a display. In other examples, the prediction notification can include a list of proposed scheduling dates and/or times for scheduling the patient. For example, the list of proposed scheduling dates and/or times can be determined and ranked based at least in part on the score of the appointment prediction model 218. Thereafter, this process proceeds to completion.

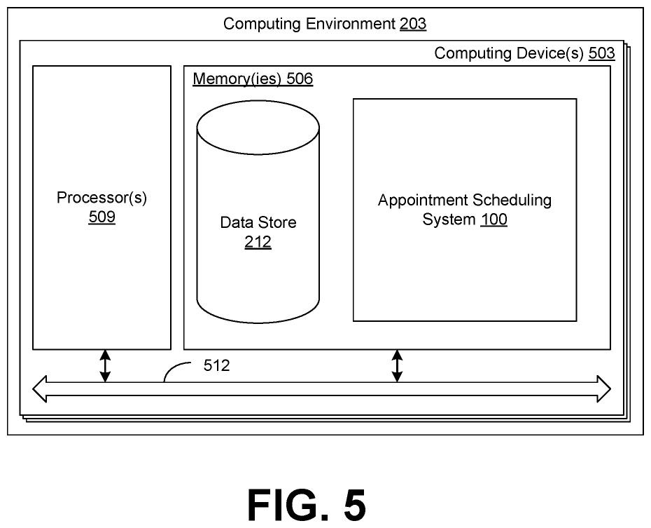

With reference to FIG. 5, shown is a schematic block diagram of the computing environment 203 according to an embodiment of the present disclosure. The computing environment 203 includes one or more computing devices 503. Each computing device 503 includes at least one processor circuit, for example, having a processor 509 and a memory 506, both of which are coupled to a local interface 512. To this end, each computing device 503 may comprise, for example, at least one server computer or like device. The local interface 512 may comprise, for example, a data bus with an accompanying address/control bus or other bus structure as can be appreciated.

Stored in the memory 506 are both data and several components that are executable by the processor 509. In particular, stored in the memory 506 and executable by the processor 509 are an appointment scheduling system 100, and potentially other applications. Also stored in the memory 506 may be a data store 212 and other data. In addition, an operating system may be stored in the memory 506 and executable by the processor 509.

It is understood that there may be other applications that are stored in the memory 506 and are executable by the processor 509 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 506 and are executable by the processor 509. In this respect, the term “executable” means a program file that is in a form that can ultimately be run by the processor 509. Examples of executable programs may be, for example, a compiled program that can be translated into machine code in a format that can be loaded into a random access portion of the memory 506 and run by the processor 509, source code that may be expressed in proper format such as object code that is capable of being loaded into a random access portion of the memory 506 and executed by the processor 509, or source code that may be interpreted by another executable program to generate instructions in a random access portion of the memory 506 to be executed by the processor 509, etc. An executable program may be stored in any portion or component of the memory 506 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 506 is defined herein as including both volatile and nonvolatile memory and data storage components. Volatile components are those that do not retain data values upon loss of power. Nonvolatile components are those that retain data upon a loss of power. Thus, the memory 506 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 509 may represent multiple processors 509 and/or multiple processor cores and the memory 506 may represent multiple memories 506 that operate in parallel processing circuits, respectively. In such a case, the local interface 512 may be an appropriate network that facilitates communication between any two of the multiple processors 509, between any processor 509 and any of the memories 506, or between any two of the memories 506, etc. The local interface 512 may comprise additional systems designed to coordinate this communication, including, for example, performing load balancing. The processor 509 may be of electrical or of some other available construction.

Although the appointment scheduling system 100, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowcharts of FIGS. 3 and 4 show the functionality and operation of an implementation of portions of the appointment scheduling system 100. If embodied in software, each block may represent a module, segment, or portion of code that comprises program instructions to implement the specified logical function(s). The program instructions may be embodied in the form of source code that comprises human-readable statements written in a programming language or machine code that comprises numerical instructions recognizable by a suitable execution system such as a processor 509 in a computer system or other system. The machine code may be converted from the source code, etc. If embodied in hardware, each block may represent a circuit or a number of interconnected circuits to implement the specified logical function(s).

Although the flowcharts of FIGS. 3 and 4 show a specific order of execution, it is understood that the order of execution may differ from that which is depicted. For example, the order of execution of two or more blocks may be scrambled relative to the order shown. Also, two or more blocks shown in succession in FIGS. 3 and 4 may be executed concurrently or with partial concurrence. Further, in some embodiments, one or more of the blocks shown in FIGS. 3 and 4 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein, including the appointment scheduling system 100, that comprises software or code can be embodied in any non-transitory computer-readable medium for use by or in connection with an instruction execution system such as, for example, a processor 509 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the appointment scheduling system 100, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing device 503, or in multiple computing devices 503 in the same computing environment 203.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, the following is claimed:

1. A system, comprising:
at least one computing device; and
an appointment scheduling application executable in the at least one computing device, wherein when executed the appointment scheduling application causes the at least one computing device to at least:
receive a request from a scheduler client device requesting a likelihood of a patient showing up for a particular appointment;
obtain a plurality of model inputs based at least in part on request, the plurality of model inputs being extracted from at least patient data and appointment data;
apply the plurality of model inputs to an appointment prediction model trained using historical appointment data, an output of the appointment prediction model indicating a prediction of whether the patient will show or fail to show to the particular appointment;
generate a notification comprising the prediction; and
send the notification to the scheduler client device.

2. The system of claim 1, wherein the appointment prediction model is trained for a given type of healthcare clinic such that a first appointment prediction model is trained for a first type of healthcare clinic and a second appointment prediction model is trained for a second type of healthcare clinic.

3. The system of claim 1, wherein the appointment prediction model is trained using a supervised machine learning model.

4. The system of claim 1, wherein the plurality of model inputs comprise at least one of: demographic data, appointment type, lifestyle data of the patient, family data, patient address data, clinic address data, insurance data, education data, occupation data, method of transportation data, an appointment length time, weather data, or traffic data.

5. The system of claim 1, wherein, when executed, the appointment scheduling application further causes the at least one computing device to at least generate an appointment recommendation based at least in part on the output of the appointment prediction model, the appointment recommendation recommending how to schedule the particular appointment.

6. The system of claim 1, wherein the appointment prediction model is a logistic regression classifier.

7. The system of claim 1, wherein generating the notification comprises generating user interface code defining a user interface or a user interface element to be rendered on a display of the scheduler client device.

8. A method, comprising:
receiving, via at least one computing device, a request from a scheduler client device requesting a likelihood of a patient showing up for a particular appointment;
obtaining, via the at least one computing device, a plurality of model inputs based at least in part on request, the plurality of model inputs being extracted from at least patient data and appointment data;
applying, via the at least one computing device, the plurality of model inputs to an appointment prediction model trained using historical appointment data, an output of the appointment prediction model indicating a prediction of whether the patient will show or fail to show to the particular appointment;
generating, via the at least one computing device, a notification comprising the prediction; and
sending, via the at least one computing device, the notification to the scheduler client device.

9. The method of claim 8, wherein the appointment prediction model is trained for a given type of healthcare clinic such that a first appointment prediction model is trained for a first type of healthcare clinic and a second appointment prediction model is trained for a second type of healthcare clinic.

10. The method of claim 8, wherein the appointment prediction model is trained using a supervised machine learning model.

11. The method of claim 9, wherein the appointment prediction model is a logistic regression classifier.

12. The method of claim 8, wherein the plurality of model inputs comprise at least one of: demographic data, appointment type, lifestyle data of the patient, family data, patient address data, clinic address data, insurance data, education data, occupation data, method of transportation data, an appointment length time, weather data, or traffic data.

13. The method of claim 8, further comprising generating an appointment recommendation based at least in part on the output of the appointment prediction model, the appointment recommendation recommending how to schedule the particular appointment.

14. The method of claim 8, further comprising:
determining a type of healthcare clinic associated with the request; and
selecting the appointment prediction model from a plurality of appointment prediction models based at least in part on the type of healthcare clinic.

15. A non-transitory computer-readable medium embodying a program executable in at least one computing device, wherein when executed the program causes the at least one computing device to at least:
receive a request from a scheduler client device requesting a likelihood of a patient showing up for a particular appointment;
obtain a plurality of model inputs based at least in part on request, the plurality of model inputs being extracted from patient data and appointment data;
apply the plurality of model inputs to an appointment prediction model trained using historical appointment data, an output of the appointment prediction model indicating a prediction of whether the patient will show or fail to show to the particular appointment;
generate a notification comprising the prediction; and
send the notification to the scheduler client device.

16. The non-transitory computer-readable medium of claim 15, wherein the appointment prediction model is trained for a given type of healthcare clinic such that a first appointment prediction model is trained for a first type of healthcare clinic and a second appointment prediction model is trained for a second type of healthcare clinic.

17. The non-transitory computer-readable medium of claim 15, wherein the appointment prediction model is trained using a supervised machine learning model.

18. The non-transitory computer-readable medium of claim 15, wherein the appointment prediction model is a logistic regression classifier.

19. The non-transitory computer-readable medium of claim 15, wherein the plurality of model inputs comprise at least one of: demographic data, appointment type, lifestyle data of the patient, family data, patient address data, clinic address data, insurance data, education data, occupation data, method of transportation data, contact date, contact length, contact time, date appointment was made, an appointment length time, weather data, or traffic data.

20. The non-transitory computer-readable medium of claim 15, wherein when executed the program further causes the at least one computing device to at least generate an appointment recommendation based at least in part on the output of the appointment prediction model, the appointment recommendation recommending how to schedule the particular appointment.
